(19) United States
(12) Patent Application Publication
(10) Pub. No.: US 2025/0326805 A1
(43) Pub. Date: Oct. 23, 2025

(54) BAX FUSION PROTEIN WITH LIMITED HYDROPHOBIC MOTIFS THROUGH OPTOGENETIC DESIGN AND COMPOSITION FOR INHIBITING APOPTOSIS USING THE SAME

(71) Applicant: KOREA UNIVERSITY RESEARCH AND BUSINESS FOUNDATION, Seoul (KR)

(72) Inventors: Seok Chung, Seoul (KR); Dain Lee, Seoul (KR); Dongwoo Oh, Seoul (KR); Hyunjun Bae, Seoul (KR); Jinchul Ahn, Seoul (KR); Seok-Hyeon Kang, Seoul (KR); JuHee Kim, Seoul (KR)

(21) Appl. No.: 19/186,503

(22) Filed: Apr. 22, 2025

(30) Foreign Application Priority Data
Apr. 23, 2024 (KR) 10-2024-0054166

(51) Int. Cl. C07K 14/47; A61K 38/00; C07K 14/705

(52) U.S. Cl. CPC C07K 14/4703; C07K 14/705; A61K 38/00; C07K 2319/60

(57) ABSTRACT

The present invention relates to a Bax fusion protein with a limited hydrophobic motif through optogenetic design and a composition for inhibiting apoptosis using the same. More specifically, the present invention relates to a Bax fusion protein to which an optogenetic protein that responds to light is bound, wherein the optogenetic protein is bound to an upper portion of the mitochondrial binding site of the Bax protein, and a composition for inhibiting apoptosis comprising the same. In the present invention, a Bax-Cry2 fusion protein n was confirmed to inhibit apoptosis when bound to TOMM20-Cib1, a mitochondrial membrane protein, by blue light. Therefore, it can be utilized as a composition for inhibiting apoptosis, and in particular, it can be applied to the treatment of degenerative neurological diseases through inhibition of apoptosis.

### CROSS REFERENCE TO RELATED APPLICATIONS

This application claims benefit of priority based on Korean Patent Application No. 10-2024-0054166 filed on Apr. 23, 2024, the disclosures of which are incorporated herein by reference in their entireties.

### REFERENCE TO ELECTRONIC SEQUENCE LISTING

The present application contains a Sequence Listing which has been submitted electronically in .XML format and is hereby incorporated by reference in its entirety. Said .XML copy, created of Apr. 21, 2025, is named “BP25-138US1_Sequence Listing.xml” and is 14,387 bytes in size. The sequence listing contained in this .XML file is part of the specification and is hereby incorporated by reference herein in its entirety.

### TECHNICAL FIELD

The present invention relates to a Bax fusion protein with a limited hydrophobic motif through optogenetic design and a composition for inhibiting apoptosis using the same. More specifically, the present invention relates to a Bax fusion protein to which an optogenetic protein that responds to light is bound, wherein the optogenetic protein is bound to an upper portion of the mitochondrial binding site of the Bax protein, and a composition for inhibiting apoptosis comprising the same.

### BACKGROUND ART

Optogenetics is a biological technology that can control cells in living tissues with light. A representative example is genetically engineering nerve cells to express ion channels that respond to light.

Optogenetics is a technology that combines optics and genetics, and can be used to control and observe the activity of individual nerve cells in living tissues, even freely moving animals, and to confirm in real time what effects the control of nerve activity causes. The main material required for optogenetics is a protein that responds to light (Deisseroth, K. ct. al., Journal of Neuroscience, 26(41): 10380˜10386, 2006). Using optogenetics, the neural activity of specific nerve cells that have been genetically classified can be selectively controlled or recorded, and the target location and time can be precisely controlled by using light.

Meanwhile, apoptosis is regulated by various proteins that contribute to various diseases ranging from cancer to neurological disorders, and in the case of degenerative neurological diseases such as Alzheimer's, Huntington's, and Parkinson's diseases, where excessive apoptosis is the main cause, research on mechanisms that can reduce the occurrence of apoptosis is necessary.

Optogenetic analogs of apoptotic proteins can be applied to control apoptosis with biochemical, spatial, and temporal precision. For example, pro-and anti-apoptotic components of the Bcl-2 protein family regulate outer mitochondrial membrane (OMM) permeability, which is important for apoptosis. Bax protein, a key factor in apoptosis, translocates from the cytoplasm to the OMM in response to apoptosis-inducing signals, and upon OMM engagement, Bax oligomerizes and participates in the formation of the mitochondrial apoptosis-induced channel (MAC), through which cytochrome c is released into the cytoplasm.

In the past, an optogenetic design method that induces apoptosis of the Bax gene was discovered (Robert M. Hughes et. al., Angew Chem Int Ed Engl., 54(41): 12064˜12068, 2015), but considering that the original mechanism of Bax is apoptosis, this result can be achieved through gene overexpression without optogenetic technology, so there is a problem that the technology is not necessary.

In addition, in the case of various brain diseases where cell regeneration does not occur well and apoptosis is the main cause, research is actively being conducted on various drugs and mechanisms to reduce rather than promote apoptosis, and therefore, research on anti-apoptotic manipulation technology that induces Bax protein, the main function of apoptosis, not to induce apoptosis is necessary.

### DISCLOSURE

### Technical Problem

Accordingly, in the present invention, an optogenetic design was performed to inhibit apoptosis by restricting the hydrophobic motif in the Bax protein, a major functional protein in apoptosis. As an optogenetically designed Bax, a Bax-Cry2 fusion protein was produced so that Cryptochrome 2 (Cry2), an optogenetic gene that responds to blue light, was co-expressed upstream of the site where Bax binds to mitochondria. In addition, when the Bax-Cry2 fusion protein bound to TOMM20-Cib1, a mitochondrial membrane protein, it was confirmed that it lost its original apoptotic function and did not induce apoptosis, thereby completing the present invention.

Accordingly, it is an object of the present invention to provide a Bax fusion protein to which an optogenetic protein that responds to light is bound.

Another object of the present invention is to provide a composition for inhibiting apoptosis or a composition for mitochondrial imaging comprising the Bax fusion protein.

### Technical Solution

To achieve the above-mentioned purpose, the present invention provides an optogenetic Bax fusion protein to which a cryptochrome 2 (Cry2) protein is bound, characterized in that the cryptochrome 2 protein is bound to an upper portion of the mitochondrial binding site of the Bax protein.

In a preferred embodiment of the present invention, the optogenetic Bax fusion protein may include the cryptochrome 2 protein represented by the amino acid sequence of SEQ ID NO: 2 between the 171st (Q, Glutamine) to 172nd (T, Threonine) sequence of the Bax protein represented by the amino acid sequence of SEQ ID NO: 1.

In another preferred embodiment of the present invention, the optogenetic Bax fusion protein, to which the cryptochrome 2 protein is bound, can be represented by the amino acid sequence of SEQ ID NO: 3.

In another preferred embodiment of the present invention, the optogenetic Bax fusion protein may additionally comprise a fluorescent protein at the C-terminal portion.

In another preferred embodiment of the present invention, the fluorescent protein may be a green fluorescent protein (GFP), a yellow fluorescent protein (YFP), a red fluorescent protein (RFP), an orange fluorescent protein (OFP), a cyan fluorescent protein (CFP), a blue fluorescent protein (BFP), a far-red fluorescent protein, or a tetracystein motif, and preferably, the red fluorescent protein may be mCherry (Monomeric derivative of DsRed fluorescent protein).

In another preferred embodiment of the present invention, the optogenetic Bax fusion protein can inhibit apoptosis.

In another preferred embodiment of the present invention, the apoptosis may be inhibited by binding of cryptochrome 2 protein and mitochondrial membrane protein TOMM20-Cib1 by blue light.

To achieve other purposes, the present invention provides a composition for mitochondrial imaging comprising the optogenetic Bax fusion protein as an active ingredient.

In addition, the present invention provides a composition for inhibiting apoptosis comprising the optogenetic Bax fusion protein as an active ingredient.

In addition, the present invention provides a pharmaceutical composition for preventing or treating a degenerative neurological disease caused by excessive apoptosis, comprising the optogenetic Bax fusion protein as an active ingredient.

In a preferred embodiment of the present invention, the degenerative neurological disease may be selected from the group consisting of Parkinson's disease, Alzheimer's disease, Huntington's disease, amyotrophic lateral sclerosis, multiple sclerosis, Creutzfeldt-Jakob disease, Lou Gehrig's disease, spinal muscular dystrophy, Niemann-Pick disease, synucleinopathy, and dementia.

### Advantageous Effects

In the present invention, a Bax-Cry2 fusion protein was produced so that the optogenetic gene Cryptochrome2 (Cry2), which responded to blue light, was co-expressed upstream of the site where Bax bound to mitochondria. The Bax-Cry2 fusion protein of the present invention was confirmed to inhibit apoptosis when bound to TOMM20-Cib1, a mitochondrial membrane protein, by blue light. Therefore, it can be utilized as a composition for inhibiting apoptosis, and in particular, it can be applied to the treatment of degenerative neurological diseases through inhibition of apoptosis.

### DESCRIPTION OF DRAWINGS

FIG. 1 is a schematic diagram comparing the Bax-Cry2 fusion protein (DBT) of the present invention with the conventional Bax-Cry2 fusion protein (HBT).

FIG. 2 is an image showing the binding of Bax-Cry2 fusion protein (DBT) and conventional Bax-Cry2 fusion protein (HBT) to TOMM20-Cib1 protein on the mitochondrial membrane was induced and confirmed by irradiating blue light for 1000 ms at 5-minute intervals for 180 minutes prior to irradiation with the blue light, and at the same time, the shape change of the cell was tracked.

FIG. 3 is an image of colocalization observed to confirm the effect of (a) Bax-Cry2 fusion protein (DBT) and (b) conventional Bax-Cry2 fusion protein (HBT) on the rate of binding of the mitochondrial membrane of intracellular Bax protein, and (c) data confirming the colocalization rate of each protein.

FIG. 4A is data confirming the degree of Bax protein aggregation, FIG. 4B is data confirming the number and shape of nuclei, and FIG. 4C is data confirming the size of mitochondria. These are data confirming the degree of apoptosis according to treatment with Bax-Cry2 fusion protein (DBT) and conventional Bax-Cry2 fusion protein (HBT).

FIG. 5 shows data confirming the degree of apoptosis when Bax-Cry2 fusion protein (HBT) was administered alone, when Bax-Cry2 fusion protein (HBT)+cisplatin was co-administered, and when Bax-Cry2 fusion protein (DBT)+cisplatin was co-administered.

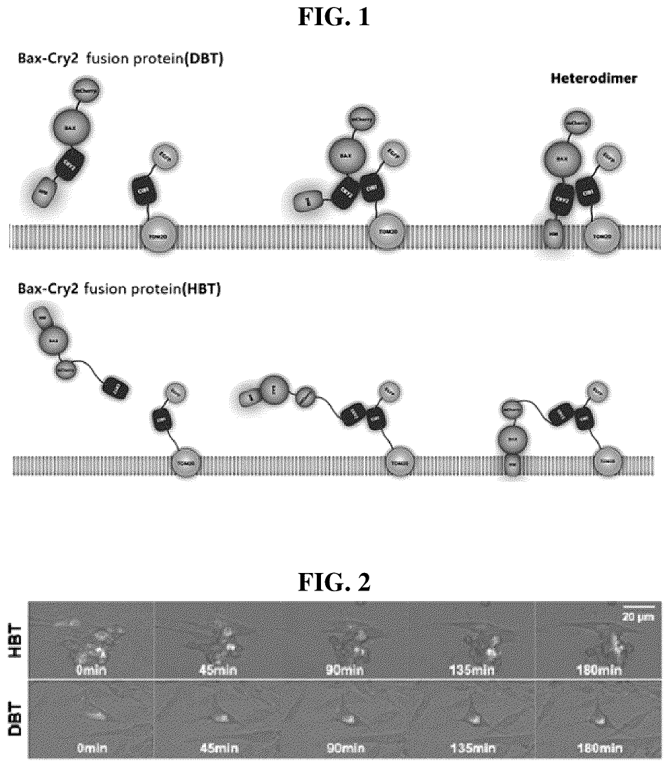

### MODE OF THE INVENTION

Hereinafter, the present invention will be described in detail.

### Optogenetically Designed Bax Protein

In one aspect, the present invention relates to an optogenetic Bax fusion protein to which a cryptochrome 2 (Cry2) protein is bound, wherein the cryptochrome 2 protein is bound above the mitochondrial binding site of the Bax protein.

In the present invention, the optogenetic Bax fusion protein may include the cryptochrome 2 protein represented by the amino acid sequence of SEQ ID NO: 2 between the 171st (Q, Glutamine) to 172nd (T, Threonine) sequence of the Bax protein represented by the amino acid sequence of SEQ ID NO: 1.

In the present invention, the optogenetic Bax fusion protein, to which the cryptochrome 2 protein is bound, can be represented by the amino acid sequence of SEQ ID NO: 3.

In the present invention, the optogenetic Bax fusion protein may additionally include a fluorescent protein at the C-terminal portion.

The fluorescent protein may be a green fluorescent protein (GFP), a yellow fluorescent protein (YFP), a red fluorescent protein (RFP), an orange fluorescent protein (OFP), a cyan fluorescent protein (CFP), a blue fluorescent protein (BFP), a far-red fluorescent protein, or a tetracystein motif, and preferably, the red fluorescent protein may be It could be mCherry (Monomeric derivative of DsRed fluorescent protein).

The cryptochrome 2 protein of the present invention is a type of protein found in plants, and is a protein that reacts to blue light with a wavelength of about 450 to 550 nm. Cryptochrome 2 protein has a property of changing its structure and becoming activated or binding to other proteins when exposed to blue light, and is therefore being used in various optogenetic technologies.

The optogenetic Cry2-Bax fusion protein manufactured in the present invention can inhibit apoptosis by causing the binding of cryptochrome 2 protein and mitochondrial membrane protein TOMM20-Cib1 by blue light.

In a specific embodiment of the present invention, in order to interfere with the mechanism by which Bax protein binds to mitochondria and induces apoptosis, Cryptochrome 2 (Cry2), an optogenetic gene that responds to blue light, was designed to be co-expressed above the hydrophobic motif region, which is the site where Bax protein binds to mitochondria. Based on the binding of Cry2 and Cryptochrome-interacting basic-helix-loop-helix1 (Cib1), it was confirmed that when the optogenetically designed Bax-Cry2 binds to TOMM20-Cib1, a mitochondrial membrane protein, it loses its original apoptosis function and does not induce apoptosis.

That is, the optogenetic Bax fusion protein of the present invention can be utilized not only as an imaging composition for observing mitochondria in real time, but also in various other ways, such as a composition for inhibiting apoptosis, and a pharmaceutical composition for preventing or treating degenerative neurological diseases caused by excessive apoptosis.

### Imaging of Mitochondria

Accordingly, in another aspect, the present invention relates to a composition for mitochondrial imaging comprising the optogenetic Bax fusion protein as an active ingredient.

In a specific embodiment of the present invention, when cells were treated with an optogenetic Bax fusion protein and then irradiated with blue light, binding of the optogenetic Bax fusion protein to the mitochondrial membrane protein, TOMM20-Cib1, was confirmed in real time, confirming that imaging of mitochondria and cells is possible using the optogenetic Bax fusion protein of the present invention.

In another aspect, the present invention relates to a kit for mitochondrial imaging comprising the optogenetic Bax fusion protein as an active ingredient.

The present kit may additionally include, but is not limited to, a buffer for cell staining, a material for cell fixation, a dish/slide for cell fixation or culture, in addition to the optogenetic Bax fusion protein.

In another aspect, the present invention relates to a method for imaging mitochondria, comprising the step of contacting a cell with the optogenetic Bax fusion protein and then irradiating it with blue light.

In the present invention, the method can observe mitochondria because the cryptochrome 2 protein of the optogenetic Cry2-Bax fusion protein and TOMM20-Cib1, a mitochondrial membrane protein, bind by blue light.

### Composition for Inhibiting Apoptosis and Therapeutic Use Thereof

In another aspect, the present invention relates to a composition for inhibiting apoptosis comprising the optogenetic Bax fusion protein as an active ingredient.

In a specific embodiment of the present invention, cells were treated with an optogenetic Bax fusion protein and irradiated with blue light, and then Bax aggregation, which is an indicator of apoptosis, the number and shape of nuclei, and the size of mitochondria were observed. As a result, it was confirmed that the Bax aggregation phenomenon was reduced by the optogenetic Bax fusion protein, the number and shape of nuclei were maintained normally, and there was little change in the size of mitochondria.

In addition, to confirm the effect of suppressing apoptosis by the optogenetic Bax fusion protein, fibroblasts were treated with the anticancer drug, cisplatin. As a result, it was confirmed that the conventional Bax-Cry2 fusion protein (HBT) alone treatment group and the HBT+drug combination treatment group induced apoptosis of fibroblasts, whereas the optogenetic Bax fusion protein (DBT) of the present invention+drug combination treatment group suppressed the apoptosis of fibroblast despite the treatment with cisplatin.

That is, the optogenetic Bax fusion protein of the present invention can be applied to various diseases caused by excessive apoptosis.

In another aspect, the present invention relates to a pharmaceutical composition for preventing or treating a neurodegenerative disease caused by excessive apoptosis, comprising the optogenetic Bax fusion protein as an active ingredient.

The above-mentioned neurodegenerative disease may be selected from the group consisting of Parkinson's disease, Alzheimer's disease, Huntington's disease, amyotrophic lateral sclerosis, multiple sclerosis, Creutzfeldt-Jakob disease, Lou Gehrig's disease, spinal amyotrophic lateral sclerosis, Niemann-Pick disease, synucleinopathy, and dementia.

Hereinafter, the present invention will be described in more detail through examples.

These examples are only intended to illustrate the present invention, and it will be apparent to those skilled in the art that the scope of the present invention is not to be construed as being limited by these examples.

### EXAMPLE 1

### Production of Optogenetic Bax Fusion Protein

The present optogenetic Bax fusion protein was designed and genetically engineered (Vectorbuilder, USA) with reference to the genetic information provided in the NIH database (Bax Gene ID: 581, Cry2 Gene ID: 839529). The present Bax fusion protein was manufactured such that the cryptochrome 2 protein represented by the amino acid sequence of SEQ ID NO: 2 was included between the 171st (Q, Glutamine) to 172nd (T, Threonine) sequence of the Bax protein represented by the amino acid sequence of SEQ ID NO: 1.

Specifically, the Bax fusion protein was represented by the amino acid sequence of SEQ ID NO: 3 and was composed of [the front part of Bax protein (Bax alpha1-8helix sequences; 1 to 171 of SEQ ID NO: 1)—linker (SEQ ID NO: 4)—cryptochrome 2 protein (SEQ ID NO: 2)—linker (SEQ ID NO: 5)—the back part of Bax protein (Bax alpha9 helix Hydrophobic motif sequences; 172 to 192 of SEQ ID NO: 1)].

As a control, a Bax-Cry2 fusion protein (HBT) represented by SEQ ID NO: 6 was prepared with reference to a conventional technique (Robert M. Hughes et. al., Angew Chem Int Ed Engl., 54(41): 12064-12068, 2015). Specifically, the Bax-Cry2 fusion protein (HBT) was composed of [Bax protein (SEQ ID NO: 7)—linker (SEQ ID NO: 8)—cryptochrome 2 protein (Shortened Cry2; SEQ ID NO: 9)—linker (SEQ ID NO: 10)].

The TOMM20-CIB1 gene, which bound to the Bax fusion protein, was purchased from Addgene (#226667).

### EXAMPLE 2

### Confirmation of Binding of Optogenetic Bax Fusion Protein and TOMM20-Cib1 Protein

In this example, it was confirmed that the Bax-Cry2 fusion protein manufactured in the present invention bound to the TOMM20-Cib1 protein of the mitochondrial membrane in response to blue light.

In the case of the real-time fluorescence image analysis in FIG. 2, the images were taken for 180 minutes, and the intracellular Bax-cry2 fusion protein (HBT or DBT) was observed as red fluorescence, and the TOMM20-Cib1 protein, which was bound to it, was observed as green fluorescence. When the two fluorescences overlap, a yellow overlapping color appeared, which observed the co-localization of the two proteins, and at the same time, changes in the cell shape were observed to indirectly confirm whether apoptosis was occurring.

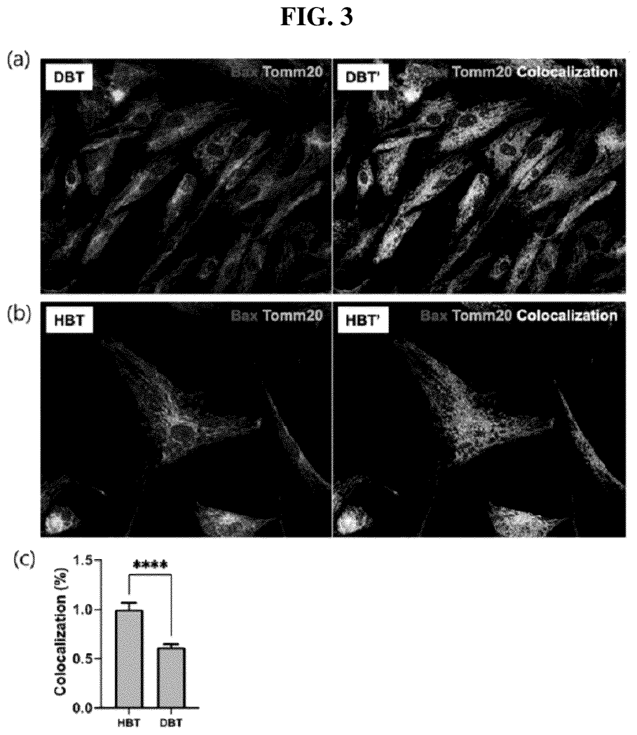

In the case of the fluorescence image analysis in FIG. 3, the cells were irradiated with blue light for 20 minutes to induce mitochondrial binding of the fusion protein, and then the cells were immediately fixed and all Bax and TOMM20 proteins within the cells were immunostained by binding each red or green fluorescent dye. This was observed under a fluorescence microscope to obtain images, thereby observing the co-localization area and confirming the effect of the intracellular fusion protein on the binding of existing intracellular proteins.

As a result, as shown in FIG. 2, after blue light irradiation, it was confirmed that the Bax-Cry2 fusion protein (DBT) and the TOMM20-Cib1 protein of the mitochondrial membrane were combined, which was also confirmed simultaneously with the conventional technique (use of HBT fusion protein). However, in the case of cells applied with the conventional technique, the cells gradually shrank and died more visibly, while in the case of cells using DBT, the cells maintained their shape relatively well.

In addition, as shown in FIG. 3, unlike the conventional Bax-Cry2 fusion protein (HBT), the Bax-Cry2 fusion protein (DBT) of the present invention did not induce apoptosis in cells to which it was applied, so that observation of intact cells/mitochondria was possible. Unlike HBT proteins, in cells to which the Bax-Cry2 fusion protein (DBT) of the present invention was applied, the colocalization rate of Bax in the mitochondrial membrane, which is a process essential for initiating apoptosis, was found to be low.

Thus, it was confirmed that the Bax-Cry2 fusion protein (DBT) restricted the co-localization of preexisting proteins for apoptosis compared to HBT, which induced apoptosis of preexisting proteins in the cell.

### EXAMPLE 3

### Confirmation of the Inhibitory Efficacy of Apoptosis by Optogenetic Bax Fusion Protein

In the present example, to confirm whether apoptosis was inhibited by the present Bax-Cry2 fusion protein, the Bax aggregation phenomenon, which is an indicator of apoptosis, the number and shape of nuclei, and the size of mitochondria were observed.

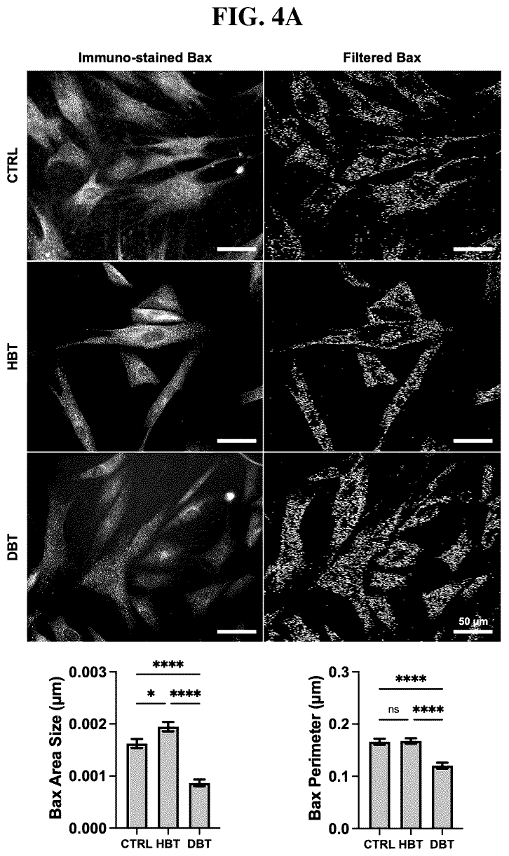

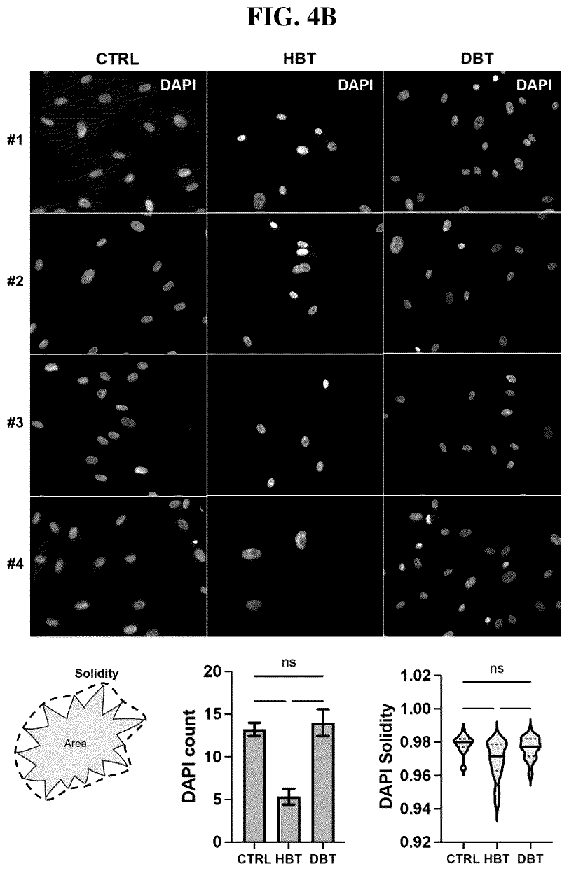

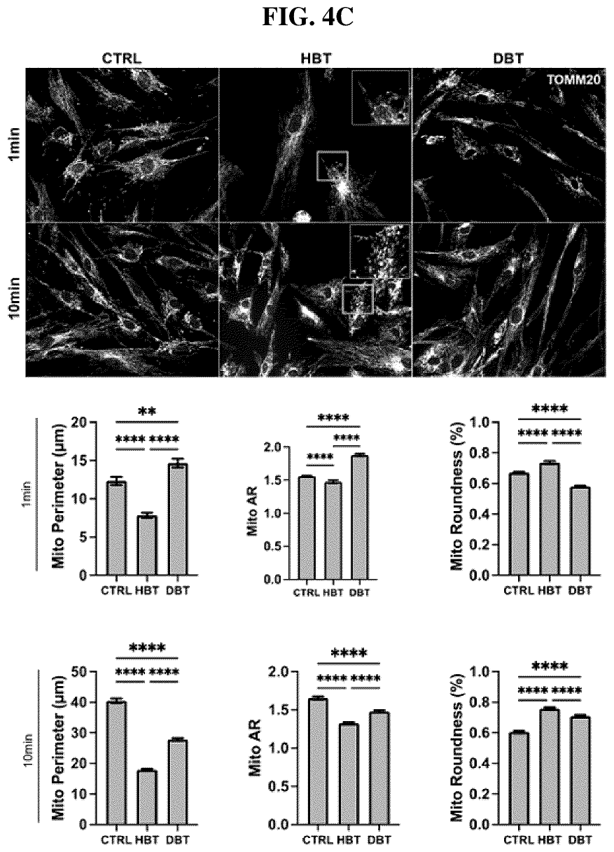

In the case of the fluorescence image analysis in FIG. 4, cells were irradiated with blue light for 20 minutes to induce mitochondrial binding of the fusion protein, then the cells were immediately fixed and all Bax proteins in the cells were immunostained with red fluorescent dye, and all Tomm20 proteins were immunostained with green fluorescent dye. By observing this with a fluorescence microscope to obtain images, we aimed to confirm the effect of the intracellular fusion protein on the binding size of existing proteins within the cell, which is a representative phenomenon that occurs during apoptosis.

In addition, by staining with DAPI (nuclear stain) dye and observing with a fluorescence microscope to obtain images, we attempted to determine the survival/death status of cells through the shape and number of nuclei within cells when the intracellular fusion protein is activated, a representative phenomenon that occurs during apoptosis.

As a result, we found that Bax aggregation was reduced, nuclear count and shape remained, and mitochondrial size was less altered by the optogenetic Bax fusion protein of the present invention, as shown in FIG. 4.

### EXAMPLE 4

### Confirmation of the Efficacy of an Optogenetic Bax Fusion Protein to Suppress Anticancer Drug-Induced Apoptosis

In the present example, in order to confirm the inhibitory effect of apoptosis by the present optogenetic Bax fusion protein, when fibroblasts were treated with the anticancer drug, cisplatin, the degree of apoptosis due to anticancer drug toxicity was confirmed.

The conditions when the conventional HBT was activated with blue light and when HBT was treated with 1 mM cisplatin and then activated with blue light were compared with the conditions when DBT was treated with 1 mM cisplatin and then activated with blue light. To this end, the cell shapes were observed for 3 hours after drug treatment to the cells using fluorescence and bright field lamps. In addition, in order to compare the apoptosis rate in other drugs, cells treated with HBT or DBT were treated with 0%, 10%, and 20% DMSO, and exposed to light for 20 minutes, and then the apoptosis signal was observed through Annexin V analysis.

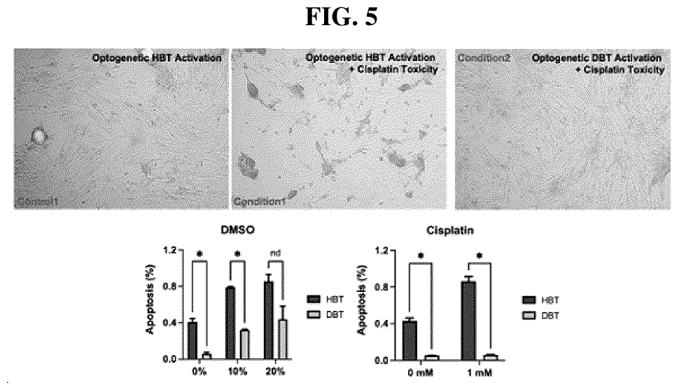

As a result, as shown in FIG. 5, it was confirmed that the conventional [Bax-Cry2 fusion protein (HBT) single treatment group] and [HBT+drug combination treatment group] induced the apoptosis of fibroblast, whereas the optogenetic [Bax fusion protein (DBT) of the present invention+drug combination treatment group] inhibited the apoptosis of fibroblast despite treatment with cisplatin.

In addition, when the degree of apoptosis after blue light irradiation of cells applied with HBT or DBT was compared under each condition where DMSO, a toxic substance, was treated at 0%, 10%, and 20% for quantitative comparison, it was confirmed that the degree of apoptosis in DBT was significantly lower in the 0% and 10% conditions, except for the 20% DMSO condition where the toxicity was very high. In a quantitative experiment after cisplatin treatment, it was also confirmed that the degree of apoptosis was significantly lower in cells applied with DBT than in HBT even when treated with 1 mM cisplatin.

That is, it was confirmed that the optogenetic Bax fusion protein of the present invention can be applied to various diseases caused by excessive apoptosis.

1. An optogenetic Bax fusion protein to which a cryptochrome 2 (Cry2) protein is bound, wherein the cryptochrome 2 protein is bound to an upper portion of the mitochondrial binding site of the Bax protein.

2. The optogenetic Bax fusion protein of claim 1, wherein the optogenetic Bax fusion protein comprises the cryptochrome 2 protein represented by the amino acid sequence of SEQ ID NO: 2 between the 171st (Q, Glutamine) to 172nd (T, Threonine) sequence of the Bax protein represented by the amino acid sequence of SEQ ID NO: 1.

3. The optogenetic Bax fusion protein of claim 1, wherein the optogenetic Bax fusion protein, to which the cryptochrome 2 protein is bound, is represented by the amino acid sequence of SEQ ID NO: 3.

4. The optogenetic Bax fusion protein of claim 1, wherein the optogenetic Bax fusion protein further comprises a fluorescent protein at the C-terminal portion.

5. The optogenetic Bax fusion protein of claim 4, wherein the fluorescent protein is selected from the group consisting of a green fluorescent protein (GFP), a yellow fluorescent protein (YFP), a red fluorescent protein (RFP), an orange fluorescent protein (OFP), a cyan fluorescent protein (CFP), a blue fluorescent protein (BFP), a far-red fluorescent protein, or a tetracystein motif.

6. The optogenetic Bax fusion protein of claim 1, wherein the optogenetic Bax fusion protein inhibits apoptosis.

7. The optogenetic Bax fusion protein of claim 1, wherein the apoptosis is inhibited by binding of cryptochrome 2 protein and mitochondrial membrane protein TOMM20-Cib1 by blue light.

8. A composition for mitochondrial imaging or a composition for inhibiting apoptosis, comprising the optogenetic Bax fusion protein of claim 1 as an active ingredient.

9. A method for preventing or treating a neurodegenerative disease caused by excessive apoptosis, comprising administering a pharmaceutical composition comprising the optogenetic Bax fusion protein of claim 1 as an active ingredient to a subject in need thereof.

10. The method for preventing or treating a neurodegenerative disease caused by excessive apoptosis of claim 9, wherein the neurodegenerative disease is selected from the group consisting of Parkinson's disease, Alzheimer's disease, Huntington's disease, amyotrophic lateral sclerosis, multiple sclerosis, Creutzfeldt-Jakob disease, Lou Gehrig's disease, spinal muscular dystrophy, Niemann-Pick disease, synucleinopathy, and dementia.


SEQUENCE LISTING (10 sequences)

SEQ ID NO: 1 - protein, 192 aa, synthetic construct
  source 1..192: note = Bax protein
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 2 - protein, 610 aa, synthetic construct
  source 1..610: note = Cryptochrome 2
MDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAPDEIVADSFEALGANTIKEPGLCPSVSSNDQQVPSAVRYNGSKRVKPEEEEERDMKKSRGFDERELFSTAESSSSSSVFFVSQSCSLASEGKNLEGIQDSSDQITTSLGKNGCK

SEQ ID NO: 3 - protein, 816 aa, synthetic construct
  source 1..816: note = Bax-Cry2(DBT)
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQGSGSGSMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAPDEIVADSFEALGANTIKEPGLCPSVSSNDQQVPSAVRYNGSKRVKPEEEEERDMKKSRGFDERELFSTAESSSSSSVFFVSQSCSLASEGKNLEGIQDSSDQITTSLGKNGCKGSGSGSTVTIFVAGVLTASLTIWKKMG

SEQ ID NO: 4 - protein, 8 aa, synthetic construct
  source 1..8: note = Linker
GSGSGSMK

SEQ ID NO: 5 - protein, 6 aa, synthetic construct
  source 1..6: note = Linker
GSGSGS

SEQ ID NO: 6 - protein, 715 aa, synthetic construct
  source 1..715: note = Bax-Cry2(HBT)
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMGGGSGGSMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAGGSDYKDDDDKARDPPVAT

SEQ ID NO: 7 - protein, 192 aa, synthetic construct; identical to SEQ ID NO: 1
  source 1..192: note = Bax(HBT)

SEQ ID NO: 8 - protein, 8 aa, synthetic construct
  source 1..8: note = Linker
GGSGGSMK

SEQ ID NO: 9 - protein, 503 aa, synthetic construct
  source 1..503: note = Shortened Cry2
MDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAGGSDYKD

SEQ ID NO: 10 - protein, 12 aa, synthetic construct
  source 1..12: note = Linker
DDDKARDPPVAT